(19) United States
(12) Patent Application Publication
(10) Pub. No.: US 2026/0079156 A1
(43) Pub. Date: Mar. 19, 2026

(54) Improved monocyte activation test

(71) Applicant: MAT RESEARCH B.V., Leiden (NL)

(72) Inventors: Koen Abraham Marijt, Oegstgeest (NL); Guillermo Freire Sanchez, Leiden (NL)

(73) Assignee: MAT RESEARCH B.V., Leiden (NL)

(21) Appl. No.: 19/108,449

(22) PCT Filed: Sep. 6, 2023
(86) PCT No.: PCT/EP2023/074390

§ 371 Date: Mar. 4, 2025

(30) Foreign Application Priority Data
Sep. 6, 2022 (EP) 22194176.8

(51) Int. Cl. G01N 33/543; G01N 33/50; G01N 33/569; G01N 33/68

(52) U.S. Cl. CPC G01N 33/54366; G01N 33/5047; G01N 33/5094; G01N 33/56972; G01N 33/6863; G01N 2333/52; G01N 2400/50

(57) ABSTRACT

The present invention is in the field of in vitro assays, particularly to detect pyrogens. The invention provides improved methods for animal-free approaches by providing an improved monocyte activation test. The improved test allows for reduced consumption of reagents and for improved throughput.

### FIELD OF THE INVENTION

The present invention is in the field of in vitro assays, particularly in the field of pyrogen and endotoxin detection. The invention provides improved methods for animal-free approaches by providing an improved monocyte activation test. The improved test allows for reduced consumption of reagents and for improved throughput.

### BACKGROUND OF THE INVENTION

Detecting the presence of pyrogens and endotoxins is highly relevant in quality and safety testing of pharmaceutical compositions and medical devices due to the capacity of pyrogens and endotoxins for causing severe adverse reactions in patients. Traditionally used detection assays include the rabbit pyrogen test (RPT) and the limulus amebocyte lysate assay (LAL), also known as bacterial endotoxin test (BET). These tests are costly, time consuming, and require the use of test animals. Further, the limulus amebocyte lysate assay is generally limited to detection of only Gram-negative bacteria and is prone to false-positives.

An alternative to the above assays is the monocyte activation test (MAT), which does not require the use of animals and is more representative of the human immune response. The MAT is a test which is governed by specific regulatory guidelines for sample preparation, testing, and analysis of results, established in the European Pharmacopoeia (Ph. Eur.; European Pharmacopoeia 10th Edition, 2019, Monograph 2.6.30, the Council of Europe). Constrained by these guidelines, the currently used protocols have low throughput, are costly, and require the use of large amount of reagents and sample volumes. Accordingly, there is still a need for improved pyrogen and endotoxin detection assays which are animal-free. Further, there is still a need for improved monocyte activation tests. There is a need for improving the throughput of monocyte activation tests. There is a need for further miniaturization of monocyte activation tests. There is a need for reducing the costs of monocyte activation tests. There is a need for reducing material consumption for monocyte activation tests.

### SUMMARY OF THE INVENTION

In a first aspect, there is provided a method for detecting a pyrogen in a sample, the method comprising the steps of:
i) providing one or more samples;
ii) contacting the sample with peripheral blood mononuclear cells (PBMCs) in an incubation medium; and
iii determining the response of the PBMCs, wherein the incubation medium has a volume of at most 175 μL per sample.

In some embodiments, the method is a monocyte activation test. In some embodiments, the incubation medium has a volume of from 20 to 150 μL, preferably of from 30 to 140 μL, more preferably of from 50 to 110 μL. In some embodiments, the contacting of step ii) is performed in a standardized 384-well plate. In some embodiments, the incubation medium comprises from 1 to 4 vol. % human medium supplement, such as about 2%. In some embodiments, the response of the PBMCs that is determined is the excretion of an inflammatory cytokine such as IL-6, IL-1beta, IL-8, TNF-alpha, MCP-1, IL-10, IFN-alpha, IFN-beta, IFN-gamma, IFN-lambda, of a prostaglandin, or of a high-mobility-group-protein. In some embodiments, the response of the PBMCs is determined by an ELISA assay. In some embodiments, the PBMCs are present at a density of at most 500×1000 cells/cm², preferably at most 250×1000 cells cm². In some embodiments, the PBMCs are present at a density of from about 10×1000 cells/cm² to about 250×1000 cells/cm², preferably of from about 50×1000 cells/cm² to about 150×1000 cells/cm², more preferably of from about 90×1000 cells/cm² to about 130×1000 cells/cm². In some embodiments, the determined response of the PBMCs for a plurality of identical samples has a coefficient of variation of at most 20%.

In some embodiments, in step i) a further control sample is provided, preferably a lipopolysaccharide sample, the control sample preferably comprising from about 0.005 to about 15 endotoxin units per mL.

In some embodiments, at least 50, preferably at least 97 samples are provided. In some embodiments, the incubation medium has a volume of from 80 to 120 μL, and the PBMCs are present at a density of from about 90 to about 130×1000 cells/cm².

In another aspect, there is provided a method for releasing a pharmaceutical composition or a medical device for use, the method comprising subjecting the pharmaceutical composition or a sample derived from the medical device to a method of the first aspect.

In another aspect, there is provided a kit of parts comprising a pyrogen or endotoxin standard, PBMCs, and one or more 384-well plates.

### DESCRIPTION OF THE INVENTION

In an aspect, there is provided a method for detecting a pyrogen in a sample, the method comprising the steps of:
i) providing one or more samples;
ii) contacting the sample with peripheral blood mononuclear cells (PBMCs) in an incubation medium; and
iii) determining the response of the PBMCs, wherein the incubation medium has a volume of at most 175 μL per sample. The method is attractive because it does not require the use of test animals. The method has a high prediction capacity.

### Pyrogens

Pyrogens are substances which can trigger an immune response in a subject by activating a cascade of immunological processes, typically characterized by an increase in the body's internal temperature outside of normal levels (fever). A biological activity of a pyrogen is its capacity to produce fever in a subject, alternatively referred to herein as its pyrogenicity. A pyrogen may be an exogenous pyrogen. “Exogenous” or “external” pyrogens refer to pyrogens originating outside of the subject's body. A pyrogen may be an endotoxin. Endotoxins, such as lipopolysaccharides (LPS), are cellular components of bacteria such as Gram-negative bacteria which constitute the main component of their outer cell walls. The presence of endotoxins in the blood stream of a subject is associated with multiple adverse symptoms, including fever, hypotension, nausea, shivering, and shock, and can lead to complications such as disseminated intravascular coagulation (DIC), endotoxin shock, and adult respiratory distress syndrome (ARDS).

A pyrogen may be a non-endotoxin pyrogen (NEP). Non-endotoxin pyrogens include microbe-associated molecular patterns (MAMPs) and pathogen-associated molecular patterns (PAMPs), with examples being flagellins, peptidoglycans, lipoproteins, lipoteichoic acid, fibroblast-stimulating lipopeptide 1, macrophage-activating lipopeptide-2, viral pyrogens, yeast pyrogens, and fungal pyrogens (e.g., yeast or fungal polysaccharides).

A pyrogen may be a product- or a process-related impurity that is present in a pharmaceutical composition or on a surface (for example of a medical device). Examples of pyrogenic impurities are chemical agents, for example polyadenylic acid, polyuridylic acid, polybionosinic acid, dinitrophenol, trinitrophenol, 4,6-dinitro-o-cresol, N-phenyl-P-naphthylamine, aldo-a-napththylamine, metals, and nanoparticles (typically <1 nm), and any other impurity that displays pyrogenicity.

A pyrogen may be a damage-associated molecular pattern (DAMP), which refers to biomolecules that are typically released by dying or damaged cells. Examples of DAMP pyrogens include byglycan, decorin, versican, hyaluronoan, fibronectin, tenascin, uric acid, S100 proteins, ATP, GTP, F-actin, cyclophilin A, histones, HMGB1, HMGN1, IL-1a, IL-33, SAP130, DNA, RNA, mtDNA, TFAM, formyl peptide, mROS, calreticulin, defensins, heat shock proteins, and any other biomolecule released by a cell that displays pyrogenicity.

A pyrogen may be a medium component of a pharmaceutical composition. Examples of such components include excipients, solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, and the like.

A pyrogen may be an endogenous pyrogen. “Endogenous” or “internal” pyrogens refer to pyrogens produced by the body of a subject following contacting with an exogenous pathogen. An endogenous pyrogen may be associated with an inflammatory response. An endogenous pyrogen may be a DAMP. Examples of endogenous pyrogens include cytokines and chemokines.

In some embodiments, the pyrogen is an exogenous pyrogen. In some embodiments, the pyrogen is an endotoxin. In some embodiments, the endotoxin is a cellular component, preferably a lipopolysaccharide, of a Gram-negative bacterium. “Gram-negative” bacterium refers to a bacterium that does not retain the crystal violet stain used in the standard Gram staining method. In some embodiments, the Gram-negative bacterium is a pathogenic bacterium. Examples of pathogenic Gram-negative bacteria are bacteria of the genera Escherichia, Salmonella, Shigella, Pseudomonas, Neisseria, Haemophilus, Bordetella, Vibrio, and the like. In some embodiments, the pyrogen is a non-endotoxin pyrogen (NEP). In some embodiments, the pyrogen is a product- or a process-related impurity that is present in a pharmaceutical composition or on a surface. In some embodiments, the pyrogen is a damage-associated molecular pattern (DAMP). In some embodiments, the pyrogen is a medium component of a pharmaceutical composition.

In some embodiments, the pyrogen is an endogenous pyrogen. In some embodiments, the endogenous pyrogen is associated with an inflammatory response. In some embodiments, the endogenous pyrogen is a damage-associated molecular pattern (DAMP). In some embodiments, the endogenous pyrogen is a cytokine. In some embodiments, the endogenous pyrogen is a chemokine.

### Step i) Provision of Samples

In step i) of the method, one or more samples are provided. A sample can be taken (derived) from an original source, for example from a product such as a pharmaceutical composition or a medical device. A sample can also be a subsample which is taken from an original sample (or from another subsample). A sample can also be, or taken from, a subsample that arises from dilution or concentration of an original sample.

A sample can also be a replicate of an original sample or of a subsample. Replicate samples are preferably identical. In cases where a sample is a replicate of an original sample or of a subsample, at least two, at least three, or at least four, preferably at least four, replicates of the original sample or of the subsample are provided. Replicates may, for example, be prepared individually or may arise from taking equal subsamples from an original sample or of a subsample. A sample is preferably a liquid sample, more preferably an aqueous sample.

A sample may be taken from a pharmaceutical composition to be tested for the presence of a pyrogen or endotoxin, for example from a therapeutic composition, diagnostic composition, or a composition for preventing a disease or condition or reducing the symptoms thereof (such as a vaccine). A pharmaceutical composition may be in any form, for example it may be a pharmaceutical composition suitable for topical, transdermal, intravenous, intramuscular, intraperitoneal, intraparenchymal, subcutaneous, intraarticular, intra-adipose tissue, oral, intrahepatic, intrasplanchnic, intra-ear, intrathoracic, intracardial, intra-ocular, or intratracheal administration, or administration via inhalation. Intra-ocular administration is preferred. In some embodiments, the pharmaceutical composition is a vaccine.

A sample may be taken from a surface to be tested for the presence of a pyrogen or endotoxin, for example from a surface of a medical device or instrument. Examples of medical devices and instruments include bedpans, cannulas, cardioverters, defibrillators, catheters, dialysers, electrocardiogramachines, enema equipment, endoscopes, gas cylinders, gauze sponges, surgical scissors, hypodermic needles, syringes, infection control equipment such as masks, gowns, face shields, and goggles, instrument sterilizers, kidney dishes, nasogastric tubes, surgical scalpels, nebulizers, ophthalmoscopes, otoscopes, pipettes, proctoscopes, radiographers, sphygmomanometers, thermometers, tongue depressors, transfusion kits, tuning forks, ventilators, watches, and the like. Such a sample may be taken, for example, by rinsing the surface to be tested with a solution (e.g., water or a buffer), collecting the rinsate, and utilizing it for sample preparation.

In some embodiments, at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, at least 132, at least 164, at least 196, at least 228, at least 260, at least 292, at least 324, at least 356, or at least 384 samples are provided. In some embodiments, at least 50, preferably at least 97 samples are provided. In some embodiments, at least 96 samples are provided. In some embodiments, at least 384 samples are provided. Advantageously the method according to the invention can be practiced using a 384-well plate.

In some embodiments, in step i) a further control sample is provided. A control sample can comprise a pyrogen (positive control) or can be free of pyrogens (negative control). Inclusion of pyrogen-positive control samples of known pyrogen concentrations can enable increased quantification accuracy. For example, using multiple samples comprising different pyrogen concentrations, a standard curve may be prepared. A control sample can be a sample from a (reference) standard. Such standards are described later herein.

In some embodiments, a control sample is an endotoxin sample, preferably a lipopolysaccharide (LPS) sample. Endotoxin is typically measured in endotoxin units per mL (EU/mL). One EU/mL is equal to approximately 0.1-0.2 ng endotoxin/mL of solution, preferably 0.15 ng/ml. In embodiments wherein the control sample is an endotoxin, preferably a lipopolysaccharide, sample, the control sample preferably comprises from about 0.005 to about 15 endotoxin units per mL. In some embodiments, the control sample comprises from about 0.005 to about 1 endotoxin units per mL. In some embodiments, the control sample comprises from about 0.008 to about 0.5 endotoxin units per mL, or from about 0.01 to about 0.4, preferably from about 0.05 to about 0.3, more preferably from about 0.1 to about 0.2 EU/mL.

Step ii) Contacting with PBMCs

In step ii), the sample or samples are contacted with peripheral blood mononuclear cells (PBMCs) in an incubation medium. In some embodiments, contacting is done with whole peripheral blood, or with a fraction thereof, containing PBMCs. In some embodiments, contacting is done with isolated PBMCs. Blood fractions containing PBMCs and isolated PBMCs may be obtained with standard methods, for example using density-gradient centrifugation.

In some embodiments, contacting is done with a PBMC cell line. In some embodiments, contacting is done with PBMCs obtained from a single donor. In some embodiments, contacting is done with PBMCs obtained from pooled whole peripheral blood from multiple donors. In cases wherein PBMCs are obtained from a single or multiple donors, the donors are preferably qualified according to standardized guidelines, more preferably as described in Sections 5-3, 5-4, 5-5, 6-3, and/or in Monograph 2.6.30 of the European Pharmacopoeia (Ph. Eur.; European Pharmacopoeia 10th Edition, 2019, the Council of Europe).

In some embodiments, contacting is done with fresh PBMCs. In preferred embodiments, contacting is done with cryo-preserved PBMCs. Cryopreservation of PBMCs may be done according to standard procedures, for example as described in standard handbooks such as Hubel, A., 2018: Preservation of Cells: A Practical Manual, 1^{st} Edition, Wiley-Blackwell, NJ, USA.

In some embodiments, the PBMCs are mammalian, preferably human. In some embodiments, the PBMCs are leukocytes. In some embodiments, the PBMCs are macrophages. In preferred embodiments, the PBMCs comprise or are monocytes, preferably mammalian monocytes, more preferably human monocytes. In some embodiments, the macrophages or monocytes are derived from pluripotent stem cells. PBMCs are well known and well characterized, and generally comprise 10-20% monocytes.

The method is preferably a monocyte activation test. More preferably the monocyte activation test is according to the guidelines laid out in Monograph 2.6.30 of the European Pharmacopoeia.

Contacting with the PBMCs in step ii) can be addition of the sample to the medium comprising the PBMCs. It can also be addition of the medium comprising the PBMCs to the sample. It may be done in any suitable receptacle, for example a microplate (with one or more wells), a tube (such as an Eppendorf tube), a flask (such as an Erlenmeyer flask), a bottle (such as a Schott bottle), a fermenter, and the like. In some embodiments, contacting is done in a standardized 96-well plate. In preferred embodiments, contacting is done in a standardized 384-well plate. Standardized well plates are widely available from commercial suppliers. Suitable standards are ANSI/SLAS standards, preferably all five of 1-2004 (R2012), 2-2004 (R2012), 3-2004 (R2012), 4-2004 (R2012), and 6-2012 (R2012).

In cases where a 96-well plate or a 384-well plate is used, a single sample can be placed in each well. The invention uniquely allows the use of 384-wells plates. Use of 384-well plates has the advantages of enabling higher detection throughput (as more samples can be tested simultaneously) and decreasing reagent requirements and overall costs. In standardized use of well-plates for MAT, a given amount of wells is required for control or reference samples. This limits the amount of wells that are available for actual test samples. A 384-well plate has a better test-to-control ratio because after allocation of wells to the required control sample, more wells remain available for test samples. In this context, the following can be a conventional allocation of wells for a 96-well plate, using 4 replicates per data point:

| | | Wells for |
|---|---|---|
| | Samples | quadruplicate |
| LPS curve | 8 | 32 |
| Test sample 1 in three concentrations | 3 | 12 |
| Test sample 1 LPS spike | 3 | 12 |
| Test sample 1 test sample 1 NEP control | 2 | 8 |
| Test sample 2 in three concentrations | 3 | 12 |
| Test sample 2 LPS spike | 3 | 12 |
| Test sample 2 test sample 1 NEP control | 2 | 8 |
| TOTAL WELLS | | 96 |

Alternately, with 2 dilutions per test sample and omitting the NEP control, the following can apply:

| | | Wells for |
|---|---|---|
| | Samples | quadruplicate |
| LPS curve | 8 | 32 |
| Test sample 1 in two concentrations | 2 | 8 |
| Test sample 1 LPS spike | 2 | 8 |
| Test sample 2 in two concentrations | 2 | 8 |
| Test sample 2 LPS spike | 2 | 8 |
| Test sample 3 in two concentrations | 2 | 8 |
| Test sample 3 LPS spike | 2 | 8 |
| Test sample 4 in two concentrations | 2 | 8 |
| Test sample 4 LPS spike | 2 | 8 |
| TOTAL WELLS | | 96 |

It should be noted that a sample of a product to be analysed can lead to multiple samples that are provided in the method for detecting a pyrogen. For instance, in the above table, a single test sample 1 results in a plurality of samples when each well is seen as comprising a provided sample. This difference will be clear from context when not explicitly indicated.

The PBMCs may be present at a specific density during the contacting step, preferably measured in cells/cm² (cm² refers to the growing area, which is preferably the area that is the cross-section of a well; preferred wells are flat-bottomed wells). The density of the PBMCs in the context of the disclosure refers to the PBMC density used per contacted sample. The skilled person can easily calculate the density in cells/cm² in a receptacle, preferably a well of a standardized 384-well plate, taking into account the growing area (cm²), the cell concentration (cells/mL) and the volume (mL), for instance by using a cell counter or by using microscopic techniques. This density is a commonly used parameter and a skilled person understands that come dead cells may be present in a population. In some embodiments, the PBMCs, preferably comprising monocytes, are present at a density of at most 500×1000 cells/cm², preferably at most 250×1000 cm².

In some embodiments, the PBMCs, preferably comprising monocytes, are present at a density of from about 10×1000 cells/cm² to about 250×1000 cells/cm², preferably of from about 50×1000 cells/cm² to about 150×1000 cells/cm², preferably of from about 60×1000 cells/cm² to about 140×1000 cells/cm², preferably of from about 70×1000 cells/cm² to about 130×1000 cells/cm², preferably of from about 80×1000 cells/cm² to about 120×1000 cells/cm², more preferably of from about 90×1000 cells/cm² to about 130×1000 cells/cm²; in some embodiments from about 105×1000 cells/cm² to about 115×1000 cells/cm². In some embodiments, the PBMCs, preferably comprising monocytes, are present at a density of 110×1000 cells/cm² or about 110×1000 cells/cm².

In some embodiments, the PBMCs, preferably comprising monocytes, are present at a density of from about 560 cells/well to about 14.000 cells/well, preferably from about 2.800 cells/well to about 8.400 cells/well, preferably from about 3360 cells/well to about 7840 cells/well, preferably from about 3920 cells/well to about 7280 cells/well, preferably from about 4480 cells/well to about 6720 cells/well, more preferably of from about 5040 cells/well to about 7280 cells/well. In some embodiments, the PBMCs, preferably comprising monocytes, are present at a density of 6160 cells/well or about 6160 cells/well. A conventional 384-well plate has a growing area of about 0.056 cm² per well. 6160 cells/well can be considered to be about 110×1000 cells/cm².

The incubation medium may be have a specific volume during the contacting step, preferably measured in mL or μL. The volume of the incubation medium in the context of the disclosure refers to the volume per sample. In the provided method, the incubation medium has a volume of at most 175 μL per sample. In some embodiments, it has a volume of at most 150 μL per sample. In some embodiments, the incubation medium has a volume of from 20 to 150 μL, preferably of from 30 to 140 μL, more preferably of from 50 to 110 μL. In some embodiments, the incubation medium has a volume of from 80 to 120 μL. In some embodiments the incubation medium has a volume of from 40 to 130 μL, preferably 60 to 120 μL, more preferably of from 70 to 115 μL, more preferably of from 75 to 105 μL, more preferably of from 85 to 105 μL.

In some embodiments, the incubation medium has a volume of from 20 to 100 μL, preferably of from 30 to 100 μL, more preferably of from 50 to 100 μL. In some embodiments, the incubation medium has a volume of from 80 to 100 μL. In some embodiments, the incubation medium has a volume of 100 μL or about 100 μL.

In some embodiments, the incubation medium has a volume of from 20 to 150 UL and the PBMCs, preferably monocytes, are present at a density of from about 10×1000 cells/cm² to about 250×1000 cells/cm². In some embodiments, the incubation medium has a volume of from 20 to 150 μL and the PBMCs, preferably monocytes, are present at a density of from about 50×1000 cells/cm² to about 150×1000 cells/cm². In some embodiments, the incubation medium has a volume of from 20 to 150 UL and the PBMCs, preferably monocytes, are present at a density of from about 90×1000 cells/cm² to about 130×1000 cells/cm².

In some embodiments, the incubation medium has a volume of from 30 to 140 UL and the PBMCs, preferably monocytes, are present at a density of from about 10×1000 cells/cm² to about 250×1000 cells/cm². In some embodiments, the incubation medium has a volume of from 30 to 140 μL and the PBMCs, preferably monocytes, are present at a density of from about 50×1000 cells/cm² to about 150×1000 cells/cm². In some embodiments, the incubation medium has a volume of from 30 to 140 UL and the PBMCs, preferably monocytes, are present at a density of from about 90×1000 cells/cm² to about 130×1000 cells/cm².

In some embodiments, the incubation medium has a volume of from 80 to 120 μL and the PBMCs, preferably monocytes, are present at a density of from about 10×1000 cells/cm² to about 250×1000 cells/cm². In some embodiments, the incubation medium has a volume of from 80 to 120 μL and the PBMCs, preferably monocytes, are present at a density of from about 50×1000 cells/cm² to about 150×1000 cells/cm². In some embodiments, the incubation medium has a volume of from 80 to 120 μL and the PBMCs, preferably monocytes, are present at a density of from about 90×1000 cells/cm² to about 130×1000 cells/cm².

In some embodiments, the incubation medium has a volume of from 50 to 110 μL and the PBMCs, preferably monocytes, are present at a density of from about 10×1000 cells/cm² to about 250×1000 cells/cm². In some embodiments, the incubation medium has a volume of from 50 to 110 μL and the PBMCs, preferably monocytes, are present at a density of from about 50×1000 cells/cm² to about 150×1000 cells/cm². In some embodiments, the incubation medium has a volume of from 50 to 110 UL and the PBMCs, preferably monocytes, are present at a density of from about 90×1000 cells/cm² to about 130×1000 cells/cm².

In some embodiments, the incubation medium has a volume of from 20 to 100 UL and the PBMCs, preferably monocytes, are present at a density of from about 10×1000 cells/cm² to about 250×1000 cells/cm². In some embodiments, the incubation medium has a volume of from 20 to 100 μL and the PBMCs, preferably monocytes, are present at a density of from about 50×1000 cells/cm² to about 150×1000 cells/cm². In some embodiments, the incubation medium has a volume of from 20 to 100 UL and the PBMCs, preferably monocytes, are present at a density of from about 90×1000 cells/cm² to about 130×1000 cells/cm².

In some embodiments, the incubation medium has a volume of from 30 to 100 UL and the PBMCs, preferably monocytes, are present at a density of from about 10×1000 cells/cm² to about 250×1000 cells/cm². In some embodiments, the incubation medium has a volume of from 30 to 100 μL and the PBMCs, preferably monocytes, are present at a density of from about 50×1000 cells/cm² to about 150×1000 cells/cm². In some embodiments, the incubation medium has a volume of from 30 to 100 μL and the PBMCs, preferably monocytes, are present at a density of from about 90×1000 cells/cm² to about 130×1000 cells/cm².

In some embodiments, the incubation medium has a volume of from 50 to 100 UL and the PBMCs, preferably monocytes, are present at a density of from about 10×1000 cells/cm² to about 250×1000 cells/cm². In some embodiments, the incubation medium has a volume of from 50 to 100 μL and the PBMCs, preferably monocytes, are present at a density of from about 50×1000 cells/cm² to about 150×1000 cells/cm². In some embodiments, the incubation medium has a volume of from 50 to 100 μL and the PBMCs, preferably monocytes, are present at a density of from about 90×1000 cells/cm² to about 130×1000 cells/cm².

In some embodiments, the incubation medium has a volume of from 80 to 100 UL and the PBMCs, preferably monocytes, are present at a density of from about 10×1000 cells/cm² to about 250×1000 cells/cm². In some embodiments, the incubation medium has a volume of from 80 to 100 μL and the PBMCs, preferably monocytes, are present at a density of from about 50×1000 cells/cm² to about 150×1000 cells/cm². In some embodiments, the incubation medium has a volume of from 80 to 100 μL and the PBMCs, preferably monocytes, are present at a density of from about 90×1000 cells/cm² to about 130×1000 cells/cm².

In some embodiments, the incubation medium has a volume of 100 μL or about 100 μL and the PBMCs, preferably monocytes, are present at a density of 110×1000 cells/cm² or about 110×1000 cells/cm².

The incubation medium may be any medium that is suitable for PBMC, preferably mammalian PBMC, more preferably human PBMC, most preferably human monocyte culturing. Preferably, the incubation medium is according to the guidelines laid out in Monograph 2.6.30 of the European Pharmacopoeia (supra). A preferred incubation medium is RPMI (Roswell Park Memorial Institute) medium, more preferably RPMI 1640 medium, which is commercially available, for example from ThermoFisher Scientific (Waltham, MA, USA). However, other suitable media may be contemplated, for example DMEM (Dulbecco's Modified Eagle's Medium), EMEM (Eagle's Minimum Essential Medium), Ham's F-10 or F-12 medium, Iscove's Modified Dulbecco's Medium (IMDM), and the like, all of which are commercially available. A further example of a suitable medium is given in the Examples section later herein.

The incubation medium may be supplemented to optimize the incubation and the subsequent PBMC response determined in step iii). Accordingly, in some embodiments, the incubation medium comprises from 0.1 to 15 vol %, preferably 0.1 to 10 vol %, more preferably 1 to 4 vol % medium supplement. Preferred medium supplement is human medium supplement. In some embodiments, the incubation medium comprises 1 or about 1 vol % medium supplement. In some embodiments, the incubation medium comprises 2 or about 2 vol % medium supplement. In some embodiments, the incubation medium comprises 3 or about 3 vol % medium supplement. In some embodiments, the incubation medium comprises 4 or about 4 vol % medium supplement. “Medium supplement” as used herein refers to additional medium components that can promote the activity, such as cytokine production, of PBMCs, preferably of human PBMCs. In some embodiments, the medium supplement is fetal bovine serum (FBS). In some embodiments, the medium supplement is human AB serum (AB).

The one or more samples contacted with peripheral blood mononuclear cells (PBMCs) are incubated for a duration sufficient for a PBMC, preferably a monocyte, response to be induced. In some embodiments, the duration of the incubation is at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, or at least 24 hours. Preferably, the duration of the incubation is at least 16 hours. In some embodiments the contacting is performed for at most 72 hours, preferably at most 36 hours, preferably at most 30 hours, more preferably at most 24 hours, still more preferably at most 18 hours, most preferably at most 16 hours.

The incubation is preferably done under cell culture, preferably human cell culture, conditions. Preferred conditions are a temperature in the range of 30-42° C. such as about 37° C. and CO₂ levels of 0-8% such as about 5%. In preferred embodiments, incubation is carried out for at least 16 hours at 37° C. and CO₂ levels of 5%. Following the incubation, the incubated sample may be used directly in step iii) or may be frozen and step iii) may be carried out at a later time point.

Step iii) Determining the Response of the PBMCs

In step iii), the response of the PBMCs, preferably mammalian PBMCs, more preferably human PBMCs, most preferably human monocytes, is determined. The response of the PBMCs can be the activation of the PBMCs. The response of the PBMCs can be the expression of a surface activation marker. Examples of surface activation markers include CD80, CD86, CD11c, CD38, CD282, and CD64. The response of the PBMCs may be the production and/or excretion, preferably excretion, of an inflammatory cytokine. Examples of inflammatory cytokines are IL-6, IL-1beta, I-6, IL-8, TNF-alpha, MCP-1, IL-10, IFN-alpha, IFN-beta, IFN-gamma, and IFN-lambda, preferably are IL-6, IL-1beta, 1-6, IL-8, TNF-alpha, MCP-1, IL-10, more preferably IL-6. The response of the PBMCs can be the production and/or excretion, preferably excretion, of a prostaglandin, an example of which is PGE2. The response of the PBMCs can be the production and/or excretion, preferably excretion, of a high-mobility-group protein, an example of which is HMGB1. The response can be the production and/or excretion of neopterin. In some embodiments, the response of the PBMCs that is determined is the expression of a surface activation marker.

In some embodiments, the response of the PBMCs that is determined is the production and/or excretion, preferably excretion, of an inflammatory cytokine such as IL-6, IL-1beta, I-6, IL-8, TNF-alpha, MCP-1, IL-10, IFN-alpha, IFN-beta, IFN-gamma, IFN-lambda, of a prostaglandin, or of a high-mobility-group-protein. The skilled person understands that determination of the PBMC response can also involve the combined determination of production and/or excretion, preferably excretion, of multiple inflammatory cytokines, prostaglandins, and/or high-mobility-group-proteins. In preferred embodiments, the response of the PBMCs that is determined is the production and/or excretion, preferably excretion, of IL-6.

Determination of the response of the PBMCs can be done directly after the contacting step, in the same or a different receptacle, or the incubation mixture may be stored, optionally frozen, and used for response determination at a different time point. In general, the response of the PBMCs correlates with the detection of a pyrogen. It may be that no response is detected, in which case no pyrogens are detected.

Determination of the response of the PBMCs may be done, for example, with quantitative PCR, flow-cytometric techniques (such as FACS analysis), or with an immunoassay, preferably an ELISA assay. The skilled person is aware of how to perform such immunoassays, descriptions of which may be found in standard handbooks such as The Immunoassay Handbook: Theory and Applications of Ligand Binding, ELISA and Related Techniques, 2013, 4^{th} Edition, Ed. Wild, D., Elsevier Science, NL, incorporated herein by reference in its entirety. Commercial ELISA kits are also available, for example the MabTech ELISAbasic IL-6 Kit (HRP, MabTech AB, Nack Strand, SE). The ELISA assay is particularly advantageous when used in the method of the invention, as it enables high-throughput testing of multiple samples.

Accordingly, in some embodiments, the response of the PBMCs is determined by an ELISA assay. In some embodiments, the ELISA assay is performed utilizing an antibody against IL-6, IL-1beta, I-6, IL-8, TNF-alpha, MCP-1, IL-10, IFN-alpha, IFN-beta, IFN-gamma, IFN-lambda, a prostaglandin, or a high-mobility-group-protein. In preferred embodiments, the ELISA assay is performed utilizing an antibody against IL-6 (anti-IL-6). Such antibodies are commercially available, for example the clone 13A5 from MabTech AB, Nack Strand, SE. In some embodiments, the ELISA assay is performed in a standardized 384-well plate. An example of application of ELISA in the context of the disclosure is provided in the Examples section later herein.

The detection method of the disclosure demonstrates low variability between measurements, particularly when a plurality of identical samples is tested. Accordingly, in some embodiments, the determined response of the PBMCs for a plurality of identical samples has a coefficient of variation of at most 30%. In some embodiments, the determined response of the PBMCs for a plurality of identical samples has a coefficient of variation of at most 25%, or 24%, 23%, 22%, or 21%. Preferably the determined response of the PBMCs for a plurality of identical samples has a coefficient of variation of at most 20%. More preferably the determined response of the PBMCs for a plurality of identical samples has a coefficient of variation of at most 15%. Still more preferably the determined response of the PBMCs for a plurality of identical samples has a coefficient of variation of at most 10%, most preferably at most 9.5%.

The detection method of the disclosure enable the detection of pyrogens or endotoxins in samples taken from various products intended for therapeutic uses or for methods of therapy, preferably therapeutic uses for or methods of therapy of humans. Examples of such products are pharmaceutical compositions, medical devices, and medical instruments as described earlier herein. If no pyrogens or endotoxins are detected in/on the products, they can then safely be released (cleared) for use. In this context, release can be seen as making available to a public while certifying the product adheres to certain (relevant) standards.

Accordingly, in an aspect, there is provided a method for releasing a product, preferably a pharmaceutical product, for use, the method comprising subjecting a sample derived from the product to the method for detecting a pyrogen or endotoxin described earlier herein. The product is preferably released only when no or low pyrogen levels are detected. This can depend on which standard is adhered to.

In some embodiments, there is provided a method for releasing a pharmaceutical composition or a medical device for use, the method comprising subjecting the pharmaceutical composition or a sample derived from the medical device to the method for detecting a pyrogen or endotoxin as described earlier herein.

The disclosure further provides a kit of parts. Preferably, the kit is suitable for carrying out the methods of the invention. The kit may comprise a pyrogen or endotoxin standard of known concentration. A preferred standard is a lipopolysaccharide (LPS) standard. Pyrogen or endotoxin standards may be prepared according to Ph. Eur. Guidelines (Monograph 2.6.30, supra) or may be obtained already prepared from commercial supplies, for example from EDQM (European Directorate for the Quality of Medicines & Healthcare; see e.g. the Ph. Eur. Reference Standards: Orders and Catalogue offered by EDQM). An example of a pyrogen standard is provided in the Examples section later herein.

The pyrogen or endotoxin standard may correspond to one or more samples. Preferably, multiple samples, each comprising a different concentration of pyrogen or endotoxin, allowing for the preparation of a standard curve. Exemplary standard concentrations in the case of an endotoxin, preferably LPS, are 0.5 EU/ml, 0.25 EU/ml, 0.125 EU/ml, 0.06 EU/ml, 0.03 EU/ml, 0.016 EU/ml, and 0.008 EU/ml. Multiple replicate (identical) samples may be provided, preferably at least two, more preferably at least three, mots preferably at least four.

The kit may comprise PBMCs, preferably monocytes, more preferably mammalian monocytes, most preferably human monocytes. Suitable PBMCs are described earlier herein.

The kit may comprise one or more 384-well plates. 384-well plates enable increased testing throughput while minimizing reagent and overall costs compared to standard pyrogen detection methods, for example compared to monocyte activation tests utilizing 96-well plates.

Accordingly, in an aspect, there is provided a kit of parts comprising a pyrogen standard, PBMCs, and one or more 384-well plates. In some embodiments, the kit is a monocyte activation test (MAT) kit. In some embodiments, the kit further comprises incubation medium, optionally in combination with (human) medium supplement as described earlier herein. In some embodiments, the (human) medium supplement is in a concentration of 1 to 4 vol %, such as about 2%.

In some embodiments, the kit is suitable for simultaneous testing of at least three distinct products, for example of distinct pharmaceutical compositions, medical devices, or medical instruments, for the presence of a pyrogen. In some embodiments, the kit is suitable for simultaneous testing of at least four distinct products. In some embodiments, the kit is suitable for simultaneous testing of at least five distinct products. In some embodiments, the kit is suitable for simultaneous testing of at least six distinct products. In some embodiments, the kit is suitable for simultaneous testing of at least seven distinct products. In some embodiments, the kit is suitable for simultaneous testing of at least eight distinct products. In some embodiments, the kit is suitable for simultaneous testing of at least nine distinct products. In some embodiments, the kit is suitable for simultaneous testing of at least ten distinct products. Testing is preferably done according to Ph. Eur. Guidelines for detection of pyrogens and endotoxins (Monograph 2.6.30, supra).

“Simultaneous” testing refers to testing of samples corresponding to the distinct products in a single plate, which allows for increased testing throughput and minimization of reagent and overall costs.

In some embodiments, the incubation medium in each sample tested per well has a volume of at most 175 μL. In some embodiments, it has a volume of at most 170, 165, 160, 155, or 150 μL. In some embodiments, the incubation medium in each sample tested per well has a volume of from 20 to 150 μL, preferably of from 30 to 140 μL, more preferably of from 40 to 130 μL, more preferably of from 50 to 120 μL, more preferably of from 60 to 115 μL, more preferably of from 70 to 110 μL, more preferably of from 80 to 105 μL, more preferably of from 90 to 100 μL. In some embodiments, the incubation medium in each sample tested per well has a volume of from 20 to 100 μL, preferably of from 30 to 100 μL, more preferably of from 50 to 100 μL. In some embodiments, the incubation medium in each sample tested per well has a volume of from 80 to 120 μL. In some embodiments, the incubation medium in each sample tested per well has a volume of from 80 to 100 μL. In some embodiments, the incubation medium in each sample tested per well has a volume of 100 μL or about 100 μL.

In some embodiments, the PBMCs are present at a density of at most 500×1000 cells/cm², preferably at most 250×1000 cells cm². In some embodiments, the PBMCs are present at a density of from about 10×1000 cells/cm² to about 250×1000 cells/cm², preferably of from about 50×1000 cells/cm² to about 150×1000 cells/cm², more preferably of from about 90×1000 cells/cm² to about 130×1000 cells/cm². In some embodiments, the PBMCs are present at a density of 110×1000 cells/cm² or about 110×1000 cells/cm².

In some embodiments, the incubation medium in each sample tested per well has a volume of from 80 to 120 μL, and the PBMCs are present at a density of from about 90 to about 130×1000 cells/cm². In some embodiments, the incubation medium in each sample tested per well has a volume of from 100 or about 100 μL, and the PBMCs are present at a density of 110×1000 cells/cm² or about 110×1000 cells/cm².

Generally, the above volume can be considered the total volume present in the well. In some embodiments, incubation medium is added in such an amount that the total volume as described above is achieved.

### General Definitions

In this document and in its claims, the verb “to comprise” and its conjugations is used in its non-limiting sense to mean that items following the word are included, but items not specifically mentioned are not excluded. In addition the verb “to consist” may be replaced by “to consist essentially of” meaning that a method, respectively component as defined herein may comprise additional step(s), respectively component(s) than the ones specifically identified, said additional step(s), respectively component(s) not altering the unique characteristic. In addition, reference to an element by the indefinite article “a” or “an” does not exclude the possibility that more than one of the element is present, unless the context clearly requires that there be one and only one of the elements. The indefinite article “a” or “an” thus usually means “at least one”.

As used herein, with “at least” a particular value means that particular value or more. For example, “at least 2” is understood to be the same as “2 or more” i.e., 2, 3, 4, 5, 6, 7, 8, 9, 10, 11, 12, 13, 14, 15, . . . , etc.

The word “about” or “approximately” when used in association with a numerical value (e.g. about 10) preferably means that the value may be the given value or 5%, preferably 1% more or less of the given value. As used herein, the term “and/or” indicates that one or more of the stated cases may occur, alone or in combination with at least one of the stated cases, up to with all of the stated cases. Various embodiments are described herein. Each embodiment as identified herein may be combined together unless otherwise indicated.

All patent applications, patents, and publications cited herein are incorporated herein by reference in their entireties. The present invention is in no way limited to the methods and materials explicitly described. The present invention is further described by the following examples which are offered for illustrative purposes only and should not be construed as limiting the scope of the invention.

### DESCRIPTION OF THE FIGURES

FIG. 1A—Effect of cell density on IL-6 production in the monocyte activation test. PBMCs were seeded in 384-well plates at indicated cell densities (×1000 cells per cm²), in a total volume of 33 μL, X-axis indicates cell density (×1000 cells per cm²). y-axis indicates IL-6 per 1000 cells. Plotted are 4 replicates (symbols) and average (horizontal line). Figure titles show total MAT volume.

FIG. 1B—as 1A but with 50 μL total MAT volume.

FIG. 1C—as 1A but with 66 μL total MAT volume.

FIG. 1D—as 1A but with 100 μL total MAT volume.

FIG. 2—Plots of absorbance values in optical density (OD) (y-axis) at each concentration of LPS in EU/ml (x-axis) for each density (×1000 cells/cm², increasing from left to right). Dotted line indicates an 0.1 OD. Depicted are the averages of three experiments with four replicates each.

FIG. 3—Signal to noise ratios (bars) plotted against density (1000 cells/cm²). Signal-to-noise ratios were calculated by dividing the OD at 0.016 EU/ml by the OD at blank.

FIG. 4—Coefficient of variation (CV) at various cell densities. Average CV % of four replicates was calculated for each concentration LPS (EU/ml) and subsequently averaged per density (1000 cells/cm²). Plots show the average (bars) and standard deviation (error bars) of three different experiments.

FIG. 5—Relative gain (y-axis) plotted as the percentage of optical density (OD) at 0.032 EU/ml LPS normalized to the optical density at 100 microliter assay volume. X-axis denotes the cell density in 1000 cells/cm². Error bars represent the standard deviation of three experiments.

FIG. 6—Average CV % at different assay volumes. Average CV % of four replicates was calculated for each concentration LPS (EU/ml) and subsequently averaged per assay volume (microliter) and density. Plots show the average (bars) and standard deviation (error bars) of three different experiments (from left to right, 55/110/220). Patterns denote cell densities in 1000 cells/cm².

FIG. 7—Absorbance (OD) plotted against concentration LPS (EU/ml). X-axis is in log scale. Gray line represents a standard curve at 110 density (×1000 cells/cm²) and in 100 microliter assay volume. Black line represents a standard curve at 220 density (×1000 cells/cm²) and in 66 microliter assay volume. Error bars indicate standard deviation of four replicates.

FIG. 8—Curve slope. Bars indicate curve slope of four-parameter logistic curve. Left: LPS standard curve at 110 density (×1000 cells/cm²) and 100 microliter assay volume. Right: LPS standard curve at 220 density (×1000 cells/cm²) and 66 microliter assay volume.

FIG. 9—Average CV %. The average CV % of four replicates was calculated for each concentration LPS (EU/ml) and subsequently averaged per volume/density combination. Left: Average CV % of LPS standard curve at 110 density (×1000 cells/cm²) and 100 microliter assay volume. Right: Average CV % of LPS standard curve at 220 density (×1000 cells/cm²) and 66 microliter assay volume.

### EXAMPLES

### Materials and Methods

### Control Preparations

Lipopolysaccharide (LPS) was obtained from EDQM (batch 5.1), and handled as instructed by the EDQM. The LPS was rehydrated by vortex mixing for 30 min in 5 mL LAL reagent water (LRW, Lonza Bioscience, Basel, CH) and diluted by vortex mixing for 3 min in LRW to a stock concentration of 10 endotoxin units per milliliter (EU/ml). LPS reference endotoxin curves (RSE) were subsequently created via serial dilution, mixed by resuspension in RPMI 1640 (Thermo-Fisher Scientific, Waltham, MA, USA).

### Cell Thawing

Vials of PBMCs (10 million PBMCs/ml) were rapidly thawed in a water bath set to 37° C. and resuspended by slowly adding pre-warmed (37° C.) RPMI medium containing 4% human medium supplement (Mediatech, Manassas, VA, USA).

### Cell Density

Samples of 0.2 EU/ml LPS were plated onto a 384-well microplate (Thermo-Fisher Scientific, Waltham MA, USA) at 50% of the final volume for various different final volumes as indicated (per experiment). Subsequently, cell suspensions were added in a 1:1 ratio at different cell concentrations as indicated (per experiment), to obtain a final concentration of human media supplement (HMS) of 2% (vol./vol.). Final concentrations of LPS corresponds to a two-fold dilution series starting at 0.1 EU/ml, obtained by resuspension in RPMI in the plate. The cells were incubated with the LPS for 20 hours+/−1 hour in an incubator (Binder (CB60), Tuttlingen, Germany) set to 37° C. and 5% CO₂, after which IL-6 concentrations were measured by ELISA as explained below. The IL-6 per 1000 cells was calculated by interpolating the measured optical density (OD) on a linear regression model of an IL-6 standard curve and dividing total IL-6 produced by the total number of cells in the well.

LPS standard curves (33 microliters at concentrations of 0.064 EU/ml, 0.032 EU/ml, 0.016 EU/ml, 0.008 EU/ml, 0.004 EU/ml) were plated onto a 384-well microplate. Cryopreserved peripheral blood mononuclear cells (PBMCs) were thawed and resuspended in RPMI medium containing 4% human medium supplement. Cell suspensions were serially diluted (dilution factor 2) at concentrations of 1514 cells per microliter, 757 cells per microliter, 378 cells per microliter, 189 cells per microliter, 94.7 cells per microliter, and 47.4 cells per microliter. 33 microliters of each cell suspension was added to the plate, resulting in a final cell density of approximately 440.000 cells/cm², 220.000 cells/cm², 110.000 cells/cm², 55.000 cells/cm², 27.500 cells/cm², and 13.700 cells/cm², respectively. Final concentrations of LPS were 0.032 EU/ml, 0.016 EU/ml, 0.008 EU/ml, 0.004 EU/ml, 0.002 EU/ml at each density. Final concentration of HMS was 2% in each well.

### MAT Incubation Volume

Samples for LPS standard curves (concentrations: 0.064 EU/ml, 0.032 EU/ml, 0.016 EU/ml, 0.008 EU/ml, 0.004 EU/ml) were plated onto a 384-well microplate at three different volumes (16.7 microliters, 33 microliters, and 50 microliters). Cryopreserved PBMCs were thawed and reconstituted in RPMI medium containing 4% human medium supplement. Cell suspensions were diluted at different cell concentrations, and added to the plate in a 1:1 ratio, resulting in final cell densities of 55.000 cells/cm², 110.000 cells/cm² and 220.000 cells/cm² at each assay volume (33 microliters, 66 microliters and 100 microliters), at a final HMS concentration of 2%.

### MAT

Samples for LPS standard curves were added to the culture plate at different volumes (in a 1:1 ratio with resuspended PBMCs and incubated for 16 hours in an incubator with 5% CO₂ at 37° C. Final concentrations of LPS were 0.5 EU/ml, 0.25 EU/ml, 0.125 EU/ml, 0.06 EU/ml, 0.03 EU/ml, 0.016 EU/ml and 0.008 EU/ml. Final concentration of HMS was 2%.

### ELISA

ELISA plates were coated with IL-6 capture antibody (clone 13A5, MabTech AB, Nack Strand, SE) at a 1:2000 concentration diluted in phosphate-buffered saline and incubated overnight at 4° C. ELISA was performed using the MabTech ELISAbasic IL-6 Kit (HRP) according to the manufacturer's protocol (MabTech). Absorbance was measured using a Thermo-Scientific absorbance plate reader at a wavelength of 450 nanometers. Background at 630 nanometers was subtracted. Supernatant (16.7 microliters) was diluted 1+1 in incubation buffer and added to the ELISA plate.

### Statistics

Statistical analysis including four-parameter/five-parameter logistic regression was performed using Graphpad Prism 8 (GraphPad Software, San Diego, CA, USA).

### Results

### LPS Response at Different Densities and Volumes at 0.1 EU/Ml

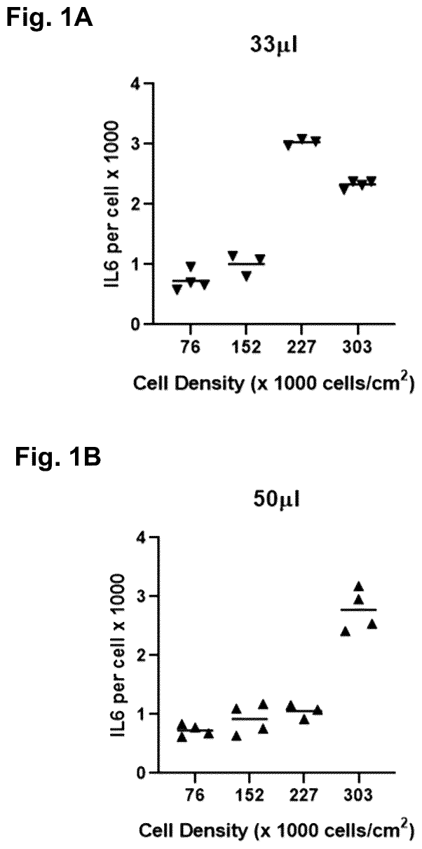

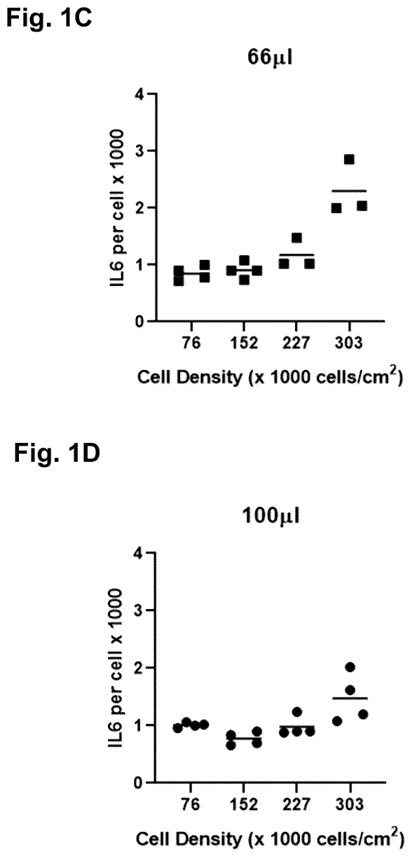

Increasing cell density showed improved IL-6 response per 1000 cells at different 384 MAT volumes at a concentration of 0.1 EU/ml, with sharp increases seen at 303 density (×1000 cells/cm²) in 50 μL, 66 μL, and 100 μL. Using 33 μL volume in the MAT, a 3-fold increase in signal was seen at 227 density (×1000 cells/cm²), compared to 152 density (×1000 cells/cm²) while a slight decrease in IL-6 per cell was seen at 303 density (×1000 cells/cm²) (FIG. 1A-D).

### Cell Density and LPS Response

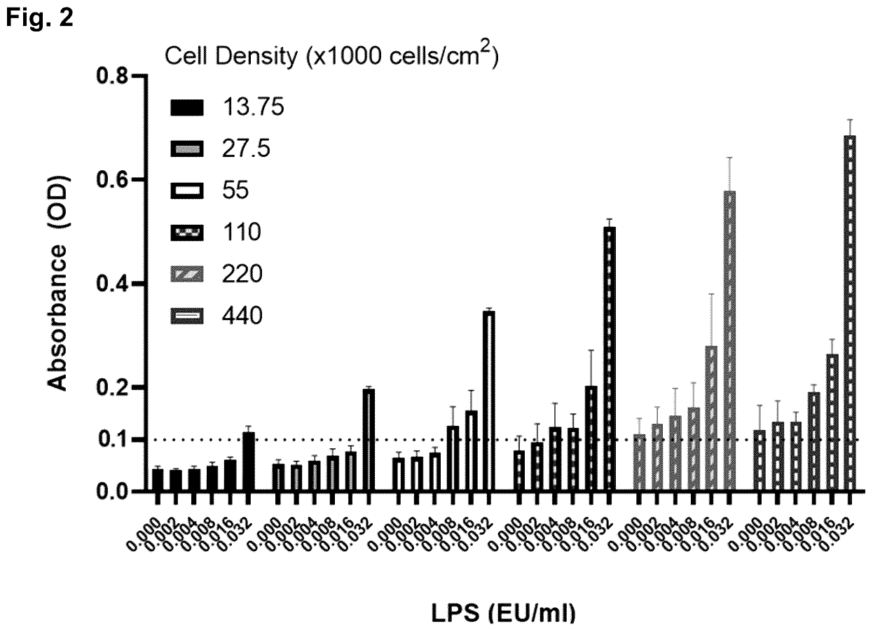

Higher densities inferred stronger LPS signal (OD) at each LPS concentration but also increased background signal (at 0.00 EU/ml) up to 0.12 OD at 440 density (×1000 cells/cm²) (FIG. 2). In addition, signal-to-noise ratio as calculated by signal at 0.032 EU/ml divided by background signal, increased when increasing density with an optimum reached at 110 density (×1000 cells/cm²) (signal-to-noise 6.4).

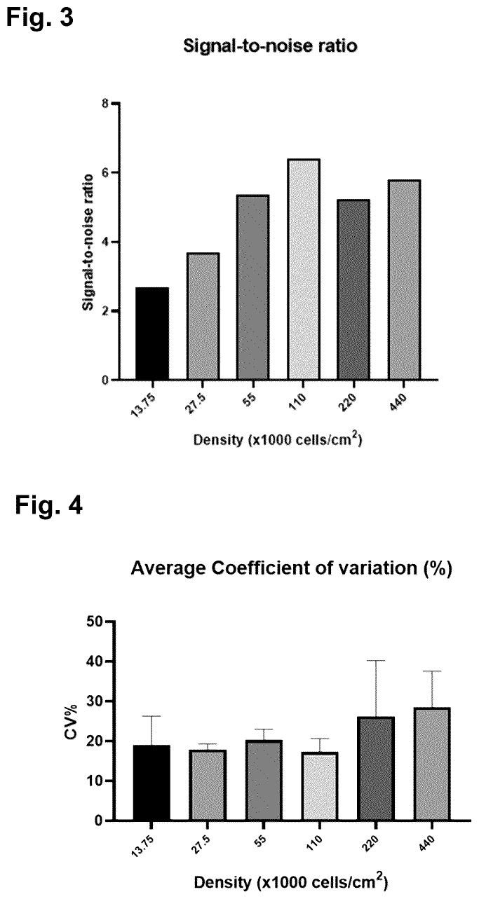

Furthermore, while LPS signal and signal to noise ratio increases with density (FIG. 3), replicate variation also increases as shown in FIG. 4, with an average CV % up to 28.5% at 440 density (×1000 cells/cm²). In between 55 and 220 density (×1000 cells/cm²) the signal-to-noise ratio was the highest among the densities tested, while the average CV % was the lowest.

### Impact of Volume on LPS Signal

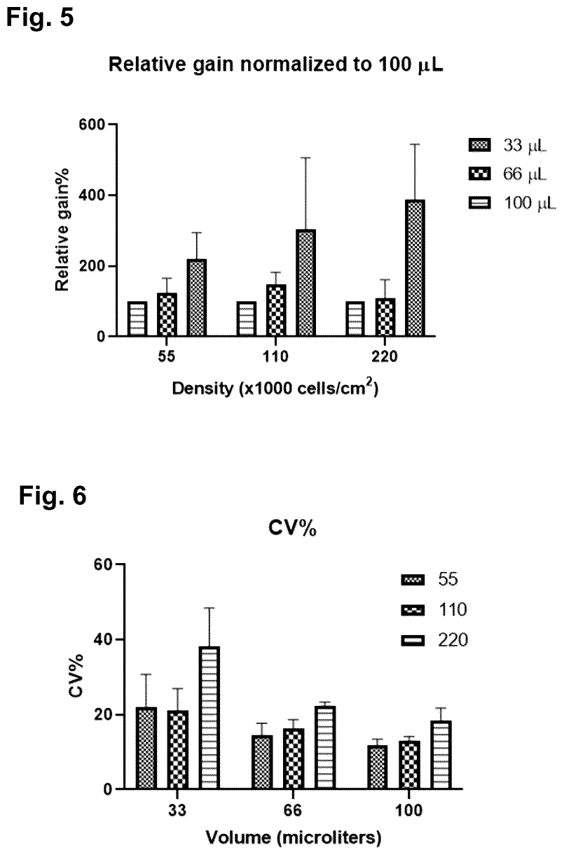

Thirty-three microliters of MAT volume showed significant increase in LPS response, as depicted in FIG. 5, showing the relative gain at 0.032 EU/ml normalized to 100 μL. Relative gain also increased when plating higher cell densities with an average relative gain of 219% at 55 density (×1000 cells per cm²), 304% at 110 density (×1000 cells per cm²), and 387% at 220 density (×1000 cells per cm²) at MAT volumes of 33 μL. However, smaller volumes showed increased variation between replicates (FIG. 6). At each density, 100 μL MAT volume showed the lowest CV % (11.8% at 55 density (×1000 cells per cm²), 13.3% at 110 density (×1000 cells per cm²) and 18.6% at 220 density (×1000 cells/cm²)). A pattern of increased CV % with increased density was observed for each assay volume, with the exception of 110 density (×1000 cells per cm²), which showed a lower CV % than 220 density (×1000 cells per cm²) in all tested assay volumes.

### LPS Standard Curves

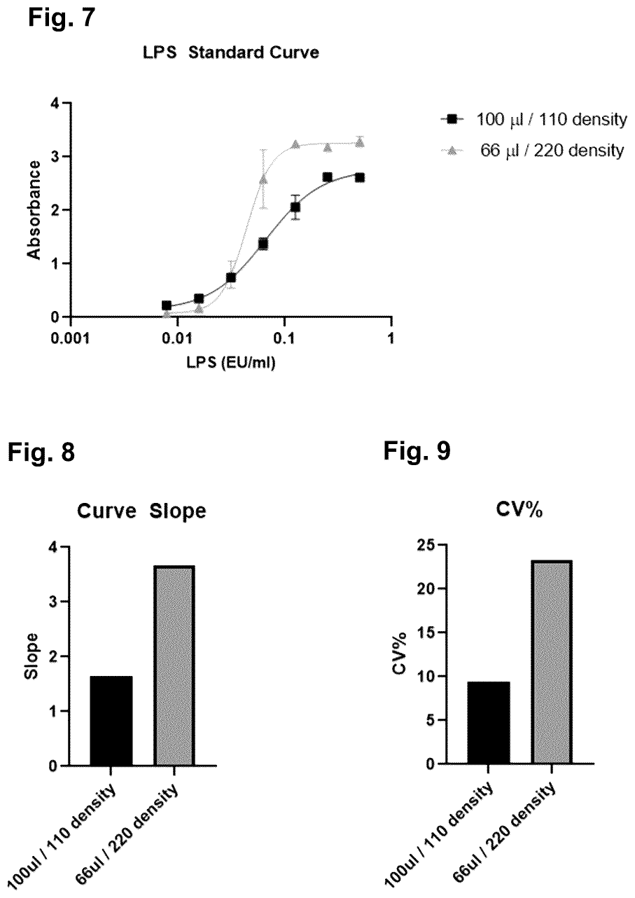

For two configurations: MAT volume at 66 μL with a cell density of 220 (×1000 cells per cm²) and MAT volume of 100 μL with a cell density of 110 (×1000 cells per cm²), an LPS standard curve was created (FIG. 7). Four-parameter logistic regression showed R2 values of 0.99 (66 μL/220 density (×1000 cells per cm²) and 0.98 (100 μL/100 density (×1000 cells per cm²)). FIG. 8 shows the curve slopes of both graphs, with approximately two-fold difference in slope between the two configurations. In addition, average CV % was markedly higher in the 66 μL/220 density (×1000 cells per cm²) MAT than the 100 μL/110 density (×1000 cells per cm²) (23.4% and 9.4%, respectively) (FIG. 9).

1. Method for detecting a pyrogen in a sample, the method comprising the steps of:
i) providing one or more samples;
ii) contacting the sample with peripheral blood mononuclear cells (PBMCs) in an incubation medium; and
iii) determining the response of the PBMCs,
wherein the incubation medium has a volume of at most 175 μL per sample.

2. The method according to claim 1, wherein the method is a monocyte activation test.

3. The method according to claim 1 or 2, wherein the incubation medium has a volume of from 20 to 150 μL, preferably of from 30 to 140 μL, more preferably of from 50 to 110 μL.

4. The method according to any one of claims 1-3, wherein the contacting of step ii) is performed in a standardised 384-well plate.

5. The method according to any one of claims 1-4, wherein the incubation medium comprises from 1 to 4 vol.-% medium supplement, such as about 2%.

6. The method according to any one of claims 1-5, wherein the response of the PBMCs that is determined is the excretion of an inflammatory cytokine such as IL-6, IL-1beta, IL-8, TNF-alpha, MCP-1, IL-10, IFN-alpha, IFN-beta, IFN-gamma, IFN-lambda, of a prostaglandin, or of a high-mobility-group-protein.

7. The method according to any one of claims 1-6, wherein the response of the PBMCs is determined by an ELISA assay.

8. The method according to any one of claims 1-7, wherein the PBMCs are present at a density of at most 500×1000 cells/cm², preferably at most 250×1000 cells cm².

9. The method according to any one of claims 1-8, wherein the PBMCs are present at a density of from about 10×1000 cells/cm² to about 250×1000 cells/cm², preferably of from about 50×1000 cells/cm² to about 150×1000 cells/cm², more preferably of from about 90×1000 cells/cm² to about 130×1000 cells/cm².

10. The method according to any one of claims 1-9, wherein the determined response of the PBMCs for a plurality of identical samples has a coefficient of variation of at most 20%.

11. The method according to any one of claims 1-10, wherein in step i) a further control sample is provided, wherein the further control sample is preferably a lipopolysaccharide sample, wherein the further control sample preferably comprises from about 0.005 to about 15 endotoxin units per mL.

12. The method according to any one of claims 1-11, wherein at least 50, preferably at least 97 samples are provided.

13. The method according to any one of claims 1-12, wherein the incubation medium has a volume of from about 80 to about 120 μL, and wherein the PBMCs are present at a density of from about 90 to about 130×1000 cells/cm².

14. Method for releasing a pharmaceutical composition or a medical device for use, the method comprising subjecting the pharmaceutical composition or a sample derived from the medical device to a method as described in any one of claims 1-13.

15. Kit of parts comprising a pyrogen or endotoxin standard, PBMCs, and one or more 384-well plates.
